(19) United States
(12) Patent Application Publication
(10) Pub. No.: US 2002/0103407 A1
(43) Pub. Date: Aug. 1, 2002

(54) Substitute natural gas production system and related method

(76) Inventor: Takefumi Hatanaka, Tokyo (JP)

Correspondence Address: JORDAN AND HAMBURG LLP, 122 EAST 42ND STREET, SUITE 4000, NEW YORK, NY, 10168, US

(21) Appl. No.: 10/058,845

(22) Filed: Jan. 28, 2002

(30) Foreign Application Priority Data
Jan. 29, 2001 (JP) 2001-59202

(51) Int. Cl. C07C 1/00

(52) U.S. Cl. 585/733

(57) ABSTRACT

A method and apparatus for producing substitute natural gas are disclosed as including a plasma reactor (PR) which has arc discharge electrodes and a large number of minute arc passages (35) formed in solid carbon materials filled in the plasma reactor. Feed water is converted into steam in steam generating zone (34A) of the plasma reactor and the steam is fed through the minute arc passages in which steam reacts the solid carbon materials in the presence of arc plasmas to produce synthesis gas. The synthesis gas is supplied to a methanation reactor (MR) which converts the synthesis gas into substitute natural gas. The arc discharge electrodes may be supplied with controlled electric power such that a H₂/CO ratio is maintained at a fixed value. The substitute natural gas may be cooled to separate condensed water, which is recycled to the plasma reactor for production of the steam therein.

### BACKGROUND OF THE INVENTION

1. Field of the Invention

This invention relates to methods and apparatus for producing substitute natural gas and, more particularly, to a method and apparatus for producing substitute natural gas using synthesis gas.

2. Description of the Related Art

Extensive research and development works have been undertaken to produce substitute natural gas using synthesis gas.

U.S. Pat. No. 4,011,058 discloses a process for the production of substitute natural gas from gasification of coal char. Carbonacious material such as coal char is gasified in the presence of air and carbon dioxide to produce a row process stream containing carbon monoxide, carbon dioxide and nitrogen. With such a gasification method, complicated steps are required to produce synthesis gas using various absorbent systems, with a resultant increase in cost for producing synthesis gas. Another drawback resides in that a plant for producing synthesis gas becomes large in size and is extremely expensive to manufacture.

U.S. Pat. No. 4,160,649 discloses a multi-stage steam reforming process for producing a substitute natural gas from kerosene boiling range hydrocarbons. This process requires complicated steps in multiple stages under various reacting conditions, requiring skilled operations and controls for the various reacting conditions. This results in increased cost for producing substituting natural gas.

U.S. Pat. No. 4,209,305 also discloses a process for making substitute natural gas from starting feedstock composed of fossil fuels such as crude oil. This process has the same issues encountered in U.S. Pat. No. 4,160,649. Also, since the crude gas is sulfur-contaminated, complicated desulferization steps must be placed, resulting in a remarkable increase in cost for purifying product gas.

U.S. Pat. No. 4,239,499 discloses a single-stage catalytic process for producing substitute natural gas from methanol and steam. The use of methanol and steam as starting materials results in a considerable increase in the cost of starting materials, causing a difficulty in reducing the production cost of the substitute natural gas.

### SUMMARY OF THE INVENTION

It is therefore an object of the present invention to provide a method and apparatus for producing high quality substitute natural gas from solid carbon materials and feed water at a remarkably low cost.

According to one aspect of the present invention, there is provided a method of producing substitute natural gas comprising the steps of: preparing a thermal plasma reactor having a thermal reactor chamber and arc discharge electrodes located in the reactor chamber; supplying solid carbon materials into the reactor chamber to form a large number of minute arc passages in the solid carbon materials; supplying electric power to the arc electrodes to create arc discharge plasmas in the minute arc passages, respectively; passing steam through the minute arc passages to create arc discharge plasmas for causing the steam to react with the solid carbon materials to produce synthesis gas containing H₂ and CO; and introducing the synthesis gas into a methanation catalyst of a methanation reactor to synthesize substitute natural gas.

According to another aspect of the present invention, there is provided a method of producing substitute natural gas comprising the steps of: preparing a thermal plasma reactor having a thermal reactor chamber and arc discharge electrodes located in the reactor chamber; supplying solid carbon materials into the reactor chamber to form a large number of minute arc passages in the solid carbon materials; supplying electric power to the arc electrodes to create arc discharge plasmas in the minute arc passages, respectively; passing plasma gas composed of steam through the minute arc passages to create arc discharge plasmas therein for causing te steam to react with the solid carbon materials to produce synthesis gas containing H₂ and CO; detecting concentrations of H₂ and CO for producing H₂ and CO detection signals; calculating a H₂/CO ratio from the H₂ and CO detection signals to produce an arc current control signal; adjusting the electric power to be supplied to the arc electrodes in response to the arc current control signal for controlling arc discharge current thereof to control the temperature of the arc discharge plasmas for thereby adjusting the H₂/CO ratio at a given value; and introducing the synthesis gas into a methanation catalyst of a methanation reactor to synthesize substitute natural gas.

According to another aspect of the present invention, there is provided a substitute natural gas production apparatus comprising: an arc plasma reactor having a solid carbon supply port, a feed water supply port, an insulating casing formed with a synthesis gas outlet, an arc plasma chamber formed in the insulating casing, arc discharge electrodes located in the arc plasma chamber, and a plurality of minute arc passages formed in solid carbon materials filled in the arc plasma chamber; feed water supply pump for supplying feed water into the feed water supply port to cause the feed water to be converted into steam; an arc power supply for supplying electric power to the arc electrodes to cause arc discharge plasmas to be generated in the minute arc passages such that the steam reacts with the solid carbon materials to produce synthesis gas containing H₂ and CO; and a methanation reactor having a methanation catalyst for converting the synthesis gas into substitute natural gas.

### BRIEF DESCRIPTION OF THE DRAWINGS

The invention, together with objects and advantages thereof, may best be understood by reference to the following description of the presently preferred embodiments together with the accompanying drawings, in which:

FIG. 1 is a schematic view of a substitute natural gas production system of a preferred embodiment according to the present invention to carry out a method of the present invention;

FIG. 2 is a cross sectional view of an arc plasma reactor forming part of the substitute gas production system shown in FIG. 1;

FIG. 3 is a block diagram of a controller shown in FIG. 1; and

FIG. 4 is a flow chart illustrating the basic sequence of operations of the substitute natural gas production system shown in FIG. 1.

### DETAILED DESCRIPTION OF THE PREFERRED EMBODIMENTS

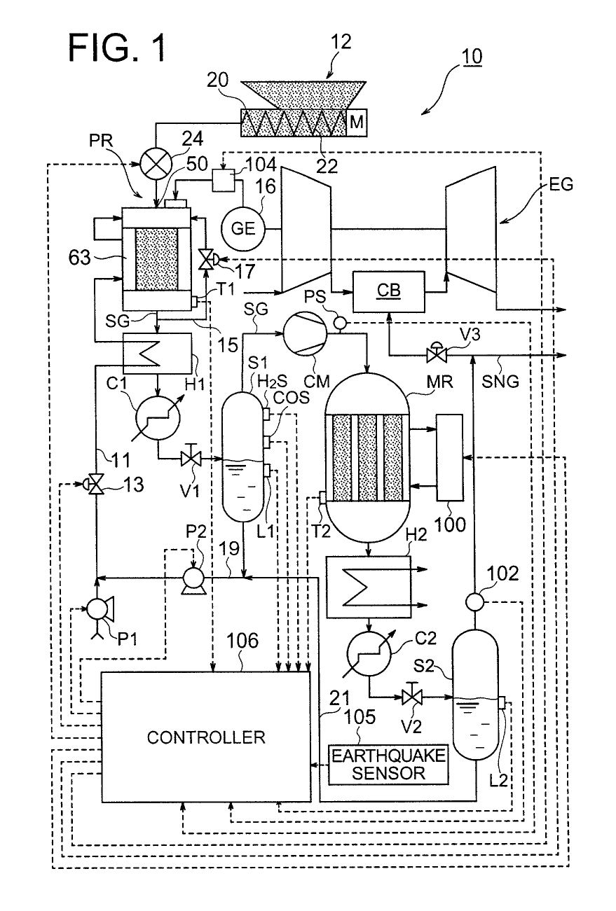

Referring to the drawings, FIG. 1 shows a substitute natural gas production system 10 of a preferred embodiment according to the present invention to which a method of the present invention is applied.

In FIG. 1, the substitute natural gas production system 10 is comprised of a solid carbon feed unit 12 which supplies solid carbon materials such as granular, particle or ball-shaped or pellet shaped graphite materials or activated carbon materials, a feed water supply line 11, a water feed pump P1 for supplying feed water to the feed water supply line 11, a flow control valve 13 which regulates the flow rate of feed water, and a thermal plasma reactor PR for converting the carbon feedstocks in the presence of water into a synthesis gas SG mainly containing hydrogen and carbon monoxide. A temperature sensor T1 is mounted to the plasma reactor PR for detecting the temperature of a plasma reactor chamber of the plasma reactor PR for producing a temperature signal, and a synthesis gas recirculation line 15 is connected between an inlet and an outlet of the plasma reactor PR for recirculating a portion of synthesis gas SG to the inlet of the plasma reactor PR. A flow control valve 17 is disposed in the synthesis gas recirculation line 15 to regulate the flow rate of synthesis gas to be recirculated to the plasma reactor PR.

A first heat exchanger H1 is located at a down stream side of the plasma reactor PR for preheating feed water in heat exchange with synthesis gas, and a cooling unit C1 is connected to the first heat exchanger H1 for cooling synthesis gas to a desired low temperature suitable for subsequent reaction. A first expansion valve V1 is connected between the cooling unit C1 and a first liquid gas separator S1 which separates moisture content from synthesis gas SG to collect condensed water. A condensed water recycle line 19 is connected to an outlet of the liquid/gas separator S1 for recycling condensed water discharged from the outlet of the first liquid/gas separator S1 through a water recycle pump P2 to the feed water supply line 11.

A first level sensor L1 is mounted to the liquid/gas separator S1 to detect the level of condensed water remaining in the first liquid gas separator S1 to produce a first level signal. A hydrogen sensor H₂S and a carbon monoxide sensor COS are also mounted to the first liquid/gas separator S1 for detecting hydrogen (H2) concentration and carbon monoxide (CO) concentration to produce a H₂ detection signal and a CO detection signal, respectively.

A compressor CM is connected to a gas outlet of the first liquid gas separator S1 for pressurizing synthesis gas SG to a value ranging from 15 to 50 atm. A pressure sensor PS is mounted to an outlet of the compressor CM for detecting the pressure of pressurized synthesis gas SG to produce a pressure signal. A methanation reactor MR is filled with a methanation catalyst which converts synthesis gas into substitute natural gas (SNG). A heater 100 supplies thermal medium to the methanation reactor MR to heat the same at a temperature range between 250 and 500° C. A temperature sensor T2 is mounted to the methanation reactor MR for detecting the reaction temperature in the methanation reactor MR to produce a reaction temperature signal.

A second heat exchanger H2 is located at an outlet of the methanation reactor MR for cooling the SNG, a cooler C2 for further cooling the SNG. A second expansion valve V2 is connected between the second heat exchanger H2 and a second liquid gas separator S2 which separates the SNG from byproduct water. A gas flow sensor 102 is connected to a gas outlet of the second liquid gas separator S2 for detecting the flow rate of the SNG to produce a SNG flow rate detection signal. A condensed water recycle line 21 is connected to the condensed water recycle line 19 to admix byproduct condensed water to feed water in the fresh feed water line 11. A branch valve V3 is provided for supplying a portion of the SNG to a combustor CB of a gas turbine engine EG which is connected to and drive an electric power generator 16. An electric power controller 104 is connected to the electric power generator 16 and is composed of an alternating three phase inverter to convert electric power output of the electric power generator 16 into a three phase alternating electric power output at a desired output voltage and a predetermined output frequency in a manner as will be described later. Further, an earth quake sensor 105 is mounted in the substitute natural gas production system to detect earth quake to produce an output signal indicative thereof.

The temperature detection signals produced by the temperature sensors T1, T2, the level signals produced by the first and second level sensors L1, L2, the H2 concentration signal produced by the hydrogen sensor H2S, the CO concentration signal produced by the CO sensor COS, the pressure detection signal produced by the pressure sensor PS, the SNG flow rate detection signal produced by the SNG flow rate sensor 102, and an earthquake detection signal produced by the earthquake sensor 105 are applied to a controller 106 by which the substitute natural gas production system 10 is controlled in operation.

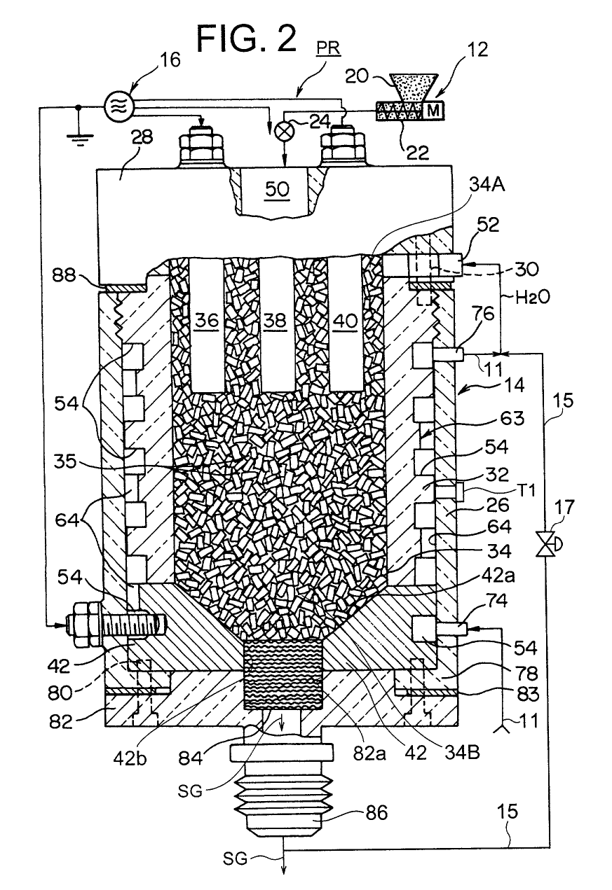

FIG. 2 shows a detailed structure of the thermal plasma reactor PR shown in FIG. 1. In FIG. 2, the thermal plasma reactor PR includes a thermal reactor unit 14 connected to the solid carbon feed unit 12, and the arc discharge power supply 16. The solid carbon feed unit 12 is comprised of a hopper 20 which stores the solid carbon materials, a screw feeder 22 and a rotary valve 24 to continuously supply the solid carbon materials at a predetermined speed. The thermal reactor unit 14 includes a cylindrical outer insulating casing 26 made of heat resistant ceramic, and an inner insulating casing 32 having a cylindrical thermal plasma reaction chamber 34. An insulating electrode holder 28 is coupled to an upper end of the inner insulating casing 32 by means of fixture bolts 30. The thermal plasma reaction chamber 34 has an upstream side formed with a steam generating zone 34A and a downstream side formed with a synthesis gas generating zone 34B. When the solid carbon materials are supplied into the thermal plasma reaction chamber 34, a large number of minute arc passages 35 are formed between adjacent gaps formed in the solid carbon materials through which large number of arc plasmas are created due to sparks in a uniform manner. When this occurs, feed water is exposed to a high temperature at the steam generating section 34A to be converted into a stream of steam. The stream of steam flows through the large number of minute arc passages 35 toward the downstream side. During such flow of stream of steam, the steam reacts with he solid carbon materials under the presence of arc plasmas to form the synthesis. When the reaction temperature in the plasma reaction chamber 34 is in a range of about 835° C., the synthesis gas contains H₂ of 47.8%, Co of 9.8%, CH₄ of 16.4%, CO₂ of 13.8%, C₂H₂ of 2.0%, C₂H₆ of 1.0%, O₂ of 2.4% and remaining hydrocarbons (CₓH_{y}) of 2.2%. At the reaction temperature of about 1000° C., the synthesis gas contains H₂ of 75.5%, Co of 13.4%, CH₄ of 2.0%, CO₂ of 7.6%, C₂H₂ of 0.3%, C₂H₆ of 0.1%, O₂ of 2.4% and remaining hydrocarbons (CₓH_{y}) of 2.2%. It will thus be seen that the hydrogen concentration in the synthesis gas increases as the reaction temperature increases and that the H₂/CO ratio can be adjusted to a suitable value for an efficient conversion of the synthesis gas into the substitute natural gas (SNG).

The insulating electrode holder 28 supports rod-like arc discharge electrodes 36, 38, 40. An annular disc shaped neutral electrode 42 is located at a lower portion of the insulating casing 32. The neutral electrode 42 has a conical surface 42a and a central opening 42b. The neutral electrode 42 is placed and supported with an electrode holder 78 formed at a bottom of the insulating casing 26 and fixed in place with fixture bolts 80. The electrode holder 28 has a carbon supply port 50 connected to the solid carbon feed unit 12. An upper portion of the outer insulating casing 26 has a feed water supply port 52 formed in the vicinity of upper areas of the arc electrodes 36, 38, 40 for introducing feed water into the steam generating section 34A. This is advantageous in that feed water serves as coolant for preventing the electrodes 36, 38, 40 from being raised to an excessively high temperature and that feed water is effectively converted into steam which serves as plasma gas for promoting generation of multiple arcs in the synthesis gas generating zone 34B. Outer peripheries of the inner casing 32 and the neutral electrode 42 are formed with cooling and heat recapturing section 63 composed of annular coolant passages 54, with the adjacent coolant passages being connected to one another through intermediate passages 54. The outer insulating 26 has an inlet 74 and an outlet 76 which communicates to one another via the coolant passages 54. Connected to the electrode holder 78 via a sealing plate 83 by means of bolts 80 is an insulating end plate 82. The neutral electrode 42 and the end plate 82 have concentric bores 42b and 82a, respectively, in which a filter 84 is received to pass synthesis gas therethrough. The end plate 82 has a synthesis gas outlet 86.

The inlet 74 is connected to the feed water line 11 and the outlet 76 is connected to the feed water supply port 52. The outlet 86 is connected to the synthesis gas recirculation line 15 which in turn is connected through the flow control valve 17 to the feed water supply port 52. Feed water is preheated in the cooling section 63 and is discharged from the outlet 76 into the feed water supply port 52. Feed water is then introduced into the steam generating section 34A to form plasma gas composed of steam. A portion of the synthesis gas emitting from the outlet 86 is delivered through the synthesis gas recirculation line 15 and the feed water supply port 52 into the thermal plasma reaction chamber 34 in which the water shift reaction takes place. Designated at 88 is a seal member.

In FIG. 2, the electrode holder 28 fixedly supports three phase rod-like electrodes 36, 38, 40 which are supplied with alternating three phase electric power from the arc discharge power supply 16. The neutral electrode 42 is connected to a neutral point of the three phase arc power discharge supply 16, which provides electric power output of output voltage in a value ranging from 30 to 240 Volts at an output frequency of 10 to 60 Hz.

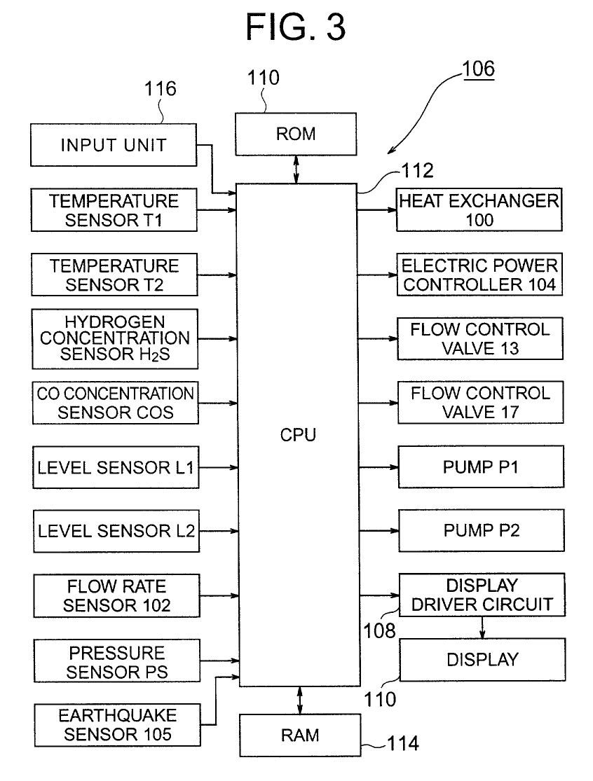

In FIG. 3, the controller 106 includes a ROM (Read On Memory) 110 which stores a control program and reference data for controlling operation of the substitute natural gas production system 10, a CPU (Central Processing Unit) 112 which executes the control program and data, and a RAM (Random Access Memory) 114 which stores preset conditions, relevant values and input information received from various sensors. The CPU 112 is comprised of an input unit 116 and is connected to the temperature sensors T1, T2, the hydrogen concentration sensor H₂S, the CO sensor COS, the level sensors L1, L2, the SNG flow rate sensor 102, the pressure sensor PS and the earth quake sensor 105 to receive relevant detection signals. The CPU 112 operates to compare these input signals with the relevant reference signals to produce various command signals in dependence on respective differences between relevant signals, with the command signals being applied to the heat exchanger 100, the electric power controller 104, the flow control valves 13, 17 and the pumps P1, P2. A display driver circuit 108 receives a display drive signal to provide a display of operating parameters, such as the detected pressures, the detected pressures, the H₂ concentration, the CO concentration, the H₂/CO ratio and the SNG flow rate, over a monitor 110.

The input unit 116 includes a start switch (not shown) and ten keys for presetting various reference data such as respective optimum operating temperatures for the thermal plasma reactor and the methanation reactor MR, the optimum H₂/Co ratio, a target pressure of the compressor CM, level values L1, L2 of condensed water and target earth quake level.

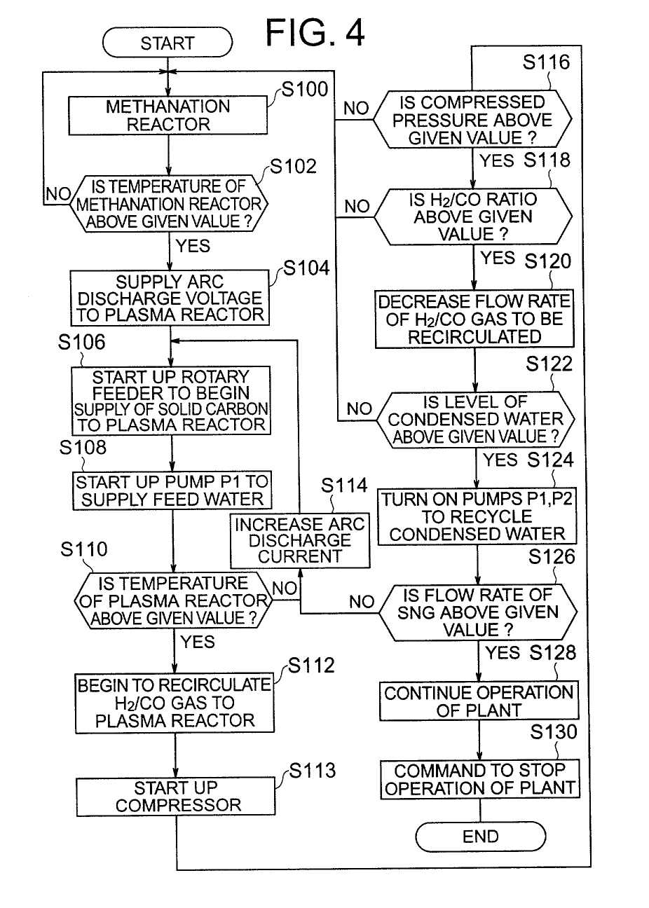

FIG. 4 shows a flow chart illustrating the basic sequence of operation for carrying out a control of the controller 106 in accordance with the substitute natural gas production method of the present invention.

When a start key is turned on, the electric power is supplied to the substitute natural gas production system. In step S100, heat medium is supplied from the heat exchanger 100 to the methanation reactor MR which is consequently heated. In step 102, the temperature of the methanation reactor MR is detected by the temperature sensor T2 and the controller discriminates whether the detected temperature exceeds a value of 250° C. In the detected temperature above 250° C., the operation goes to step S104. In contrast, when the detected temperature is below 250° C., the operation returns to step S100.

In step S104, the thermal plasma reactor PR is supplied with arc discharge voltage, and, in steps S106, 108, the rotary feeder 24 and the pump P1 are turned on to supply the solid carbon materials and feed water to the thermal plasma reactor PR. When this occurs, feed water is converted into the steam at the steam generating zone 34A in the thermal plasma reactor PR, with the steam stream flowing through the minute plasma passages 35 as plasma gas to promote generation of large number of arc discharge plasmas. During flow of steam, steam reacts with the solid carbon materials under the presence of arc discharge plasmas to produce the synthesis gas SG at the synthesis gas generating zone 35B.

In step S110, the controller 106 discriminates whether the temperature signal T1 exceeds a value of 1000° C. and, in case of “YES”, the operation goes to step S112 whereas, in case of “NO”, the operation goes to step S114.

In step S112, a portion of the synthesis gas is recirculated to the plasma reactor PR. In step S114, the electric power controller 104 increases the output frequency of the three phase electric power for thereby increasing the discharge voltage, which varies on V/F (Voltage/Frequency) pattern, to increase arc discharge current passing through the plasma reactor PR and, thereafter, the operation returns to step S106. As the arc discharge current increases, the thermal plasma temperature increases for thereby increasing the H²/Co ratio.

In step S112, when the portion of the synthesis gas is supplied to the thermal plasma reactor PR, carbon monoxide and carbon dioxide contained in the synthesis gas are reacted with steam to effectuate a water shift reaction.

In step S113, the compressor CM is turned on to compress the synthesis gas SG.

In step S116, the controller 106 discriminates whether the pressure signal PS exceeds the reference pressure of 15 atm. When the pressure signal exceeds the reference pressure, the operation goes to step S118. In contrast, if the pressure signal is below the reference pressure, then, the operation returns to step S100.

In step S118, the CPU 112 of the controller 106 calculates the H₂/CO ratio on the basis of the hydrogen concentration signal H₂S and the CO concentration signal CO, with the calculated H₂/CO ratio being compared with the reference value. If the calculated H₂/CO ratio is above a value of 3, the operation goes to step S120. In contrast, if the calculated H²/CO ratio is below the value of 3, then, the operation returns to step S100.

In step S120, the flow rate of the recirculation gas is reduced by lowering the opening degree of the flow control valve 17.

In step S122, the controller 106 discriminates whether the condensed water levels L1, L2 exceed respective reference levels. If the level signals exceed the respective reference levels, the operation goes to step S124. In contrast, if the level signals are below the respective reference levels, the operation returns to step S100.

In step S124, the pump P1 is turned off to stop the supply of feed water whereas the pump P2 is turned on. When this occurs, condensed water in the first and second gas liquid separators S1, S2 are circulated to the plasma reactor PR via the condensed water recycle lines 19, 21 and the feed water supply line 11.

In step S126, the controller 106 discriminates whether the flow rate of the SNG exceeds the reference flow rate of the SNG. If the flow rate of the SNG exceeds the given value, the operation goes to step S128. In contrast, if the flow rate of the SNG is below the given value, the operation returns to step S114 for the reasons discussed above. In step S128, the operation of the substitute natural gas production system is continued. But, if the earth quake signal exceeds a given value, then, a stop command is applied to the substitute natural gas production system for stopping the operation of the same.

Now, the operation of the substitute natural gas production system 10 is described with reference to FIG. 1. In FIG. 1, first, the heat exchanger 100 is start up to maintain the methanation reactor MR at the temperature of 250 to 500° C. During this time period, the arc discharge electric power is supplied to the arc discharge electrodes of thermal plasma reactor PR while the screw feeder 22 and the rotary valve 24 are driven to feed the solid carbon materials to the thermal plasma reactor PR. Next, the feed water supply pump P1 is driven to supply feed water to the steam generating zone 34A of the thermal plasma reaction chamber 34 from the feed water supply port 52, with feed water being exposed to the high temperature to generate plasma gas. Plasma gas flows into the large number of minute plasma passages 35, with steam reacting with the solid carbon materials at the temperature of more than 1000° C. to be converted into synthesis gas with H²/Co ratio of more than 3. Synthesis gas SG is cooled in the first heat exchanger H1 and is then further cooled in the cooler C1 to the temperature in the range between 60 to 90° C. Synthesis gas thus cooled is supplied via the shut off valve V1 to the liquid/gas separator S1 where moisture component is separated from synthesis gas SG as condensed water. When condensed water reaches the level L1, the pump P2 is driven to supply condensed water to the feed water supply line 11 via the recycle line 19 to be admixed with fresh feed water. Mixed water is preheated at the cooling section 63 of the thermal plasma reactor PR and is then supplied to the feed water supply port 52. On the other hand, synthesis gas SG is pressurized at the pressure level of about 15 to 50 atm and is introduced into the methanation reactor MR, which is maintained at the temperature of 250 to 500° C., thereby converting synthesis gas into substitute natural gas. The methanation catalyst to be filled in the reactor MR may be of any type disclosed in, for example, U.S. Pat. Nos. 4,238,371, 4,368,142, and 4,774,261 and Japanese Patent Provisional Publication No. 5-184,925. Substitute natural gas is cooled at the heat exchanger H2 and the cooler C2 and is supplied through the pressure reduction valve V2 to the liquid/gas separator S2 where condensed water is separated from substitute natural gas, with condensed water being recycled from the bottom of the liquid/gas separator S2 to the feed water supply line 11 via the recycle line 21 and the circulation pump P2 to be recycled to the thermal plasma reactor PR. Product gas SNG is supplied to outside, while a portion of product gas is supplied to the combustor CB of the electric power generator EG for generating electric power in the manner as described above.

The system and method of the present invention provides numerous advantages over the prior art practices and which includes:

(1) Feed water and solid carbon materials which are extremely low in cost can be utilized as raw materials, resulting in a remarkable reduction in production cost of SNG.

(2) The utilization of thermal plasma reactor which is small in structure but has a high operating performance enables efficient production of synthesis gas in a large volume for thereby increasing the production efficiency of SNG.

(3) Since the solid carbon materials are consumed only for producing synthesis gas and no carbon materials are used as fuels for the reformer as would required in the prior art practice, the utilization rate of the solid carbon materials is extremely high.

(4) Since the H²/CO ratio of synthesis gas can be easily adjusted to a suitable value effective for the maximum performance in producing SNG by controlling the operating temperature of the thermal plasma reactor PR, it is possible for the SNG production system to be controlled in operation to provide an optimum operating control.

(5) Although the prior art practice needs a complex process for intermittently supply air into the reformer while interrupting the synthesis gas production, the system and the method of the present invention do not require such a complex process for thereby simplifying the control in operation while enabling a remarkable reduction of production cost.

(6) In the prior art practice, since the reformer adopts the combustion method for producing synthesis gas, it is difficult for the reformer to control the operating temperature according to the operating condition of the SNG production plant at a high speed response. On the contrary, the present invention enables the thermal plasma reactor to be precisely controlled at an appropriate temperature by merely varying the voltage of the power supply to be applied to the arc discharge electrodes, providing a quick response to enable a mass production of SNG at the maximum efficiency.

(7) In the prior art practice, condensed water obtained during refining of SNG is expelled outside, causing environmental contaminants. On the contrary, condensed water is recycled as feed water to the thermal plasma reactor PR, with a resultant remarkable decrease in the amount of feed water while eliminating environmental pollution.

(8) In the reforming process of natural gas to produce synthesis gas using partial combustion method carried out in the prior art, it takes a longer rise time and a longer dwell time. In contrast, the presence of capability of instantaneously producing synthesis gas by supplying electric power to the arc discharge electrodes allows the SNG production system to be started up and terminated in operation in quick response. This is especially advantageous for an emergency stop such as earth quake.

(9) In the prior art practice, the SNG plant has a remarkably large size in a whole structure while increasing running cost, thus requiring a sizable financial investment for such a plant. In contrast, the SNG production system according to the present invention is small in size but high in operating efficiency and, therefore, there is no need for the sizable investment.

While a specific embodiment of the invention has been described in detail, it will be appreciated by those skilled in the art that various modifications and alternatives to those details could be developed in light of the overall teachings of the disclosure. Accordingly, the particular embodiment disclosed is meant to be illustrative only and not limiting to the scope of invention which is defined in appended claims.

What is claimed is:

1. A method of producing substitute natural gas comprising the steps of:
preparing a thermal plasma reactor having a thermal reactor chamber and arc electrodes located in the reactor chamber;
supplying solid carbon materials into the reactor chamber to form a large number of minute arc passages in the carbon materials;
supplying electric power to the arc electrodes to create arc discharge plasmas in the minute arc passages, respectively;
passing steam through the minute arc passages to react with the solid carbon materials under the presence of the arc discharge plasmas to produce synthesis gas containing H₂ and CO; and
introducing the synthesis gas into a methanation catalyst of a methanation reactor to synthesize substitute natural gas.

2. The method of claim 1, wherein the thermal plasma reactor has an upstream side formed with a steam generating zone and a downstream side formed with a reacting zone, and further comprising the steps of:
supplying feed water into the steam generating zone of the thermal plasma reactor to form the steam at the upstream side thereof;
cooling the substitute natural gas to separate condensed water; and
circulating the condensed water into the steam generating zone to be converted into the steam.

3. The method of claim 1, further comprising the steps of:
controlling the electric power supply to vary the temperature of the arc discharge plasma for thereby controlling a H₂/CO ratio at a given value.

4. The method of claim 1, further comprising the steps of:
circulating a portion of the synthesis gas into the thermal plasma reactor.

5. A method of producing substitute natural gas comprising the steps of:
preparing a thermal plasma reactor having a thermal reactor chamber and arc discharge electrodes located in the reactor chamber;
supplying solid carbon materials into the reactor chamber to form a large number of minute arc passages in the solid carbon materials;
supplying electric power to the arc electrodes to create arc discharge plasmas in the minute arc passages, respectively;
passing steam through the minute arc passages to create arc discharge plasmas for causing the steam to react with the solid carbon materials under the presence of the arc discharge plasmas to produce synthesis gas containing H₂ and CO;
detecting concentrations of H₂ and CO for producing H₂ and CO detection signals;
calculating a H₂/CO ratio from the H₂ and CO detection signals to produce an arc current control signal;
adjusting the electric power to be supplied to the arc electrodes in response to the arc current control signal for controlling arc discharge current thereof to control the temperature of the arc discharge plasma for thereby adjusting the H₂/CO ratio at a given value; and
introducing the synthesis gas into a methanation catalyst of a methanation reactor to synthesize substitute natural gas.

6. The method of claim 5, wherein the thermal plasma reactor has an upstream side formed with a steam generating zone and a downstream side formed with a reacting zone, and further comprising the steps of:
supplying feed water into the steam generating zone of the thermal plasma reactor to form the steam as the plasma gas at the upstream side thereof;
cooling the substitute natural gas to separate condensed water; and
circulating the condensed water into the steam generating zone to be converted into the steam.

7. A substitute natural gas production apparatus comprising:
an arc plasma reactor having a solid carbon supply port, a feed water supply port, an insulating casing formed with a synthesis gas outlet, an arc plasma chamber formed in the insulating casing, arc discharge electrodes located in the arc plasma chamber, and a plurality of minute arc passages formed in solid carbon materials filled in the arc plasma chamber;
a feed water supply pump for supplying feed water into the feed water supply port to cause the feed water to be converted into steam;
an arc discharge power supply for supplying electric power to the arc discharge electrodes to cause arc discharge plasmas to be generated in the minute arc passages such that the steam reacts with he solid carbon materials to produce synthesis gas containing H₂ and CO; and
a methanation reactor having a methanation catalyst for converting the synthesis gas into substitute natural gas.

8. The substitute natural gas production apparatus of claim 7, further comprising:
a condenser unit coupled to the methanation reactor for cooling the substitute natural gas to separate condensed water therefrom; and
a recycle line for recycling the condensed water to the arc plasma reactor to form the steam therein.

9. The substitute natural gas production apparatus of claim 7, further comprising:
a first detector located in the arc plasma reactor for detecting a H₂ concentration in the synthesis gas to produce a H2 detection signal;
a second detector located in the arc plasma reactor for detecting a CO concentration in the synthesis gas to produce a CO detection signal;
a controller responsive to the H2 and CO detection signals for producing an electric power control signal; and
an electric power controller responsive to the electric power control signal for controlling the electric power supply to be supplied to the arc discharge electrodes for adjusting the temperature of the arc discharge plasmas such that a H₂/CO ratio is maintained at a given value.
